(19) United States
(12) Patent Application Publication
(10) Pub. No.: US 2008/0125647 A1
(43) Pub. Date: May 29, 2008

(54) Antenna System For Monitoring Of A Target Area

(75) Inventors: Bengt Rosengren, Hovås (SE); Bo Lennernas, Uddevalla (SE); Roman Iustin, Molndal (SE); Tomas Gustafsson, Molndal (SE)

Correspondence Address: BIRCH STEWART KOLASCH & BIRCH, PO BOX 747, FALLS CHURCH, VA, 22040-0747, US

(73) Assignee: Micropos Medical AB, Goteborg (SE)

(21) Appl. No.: 11/793,049

(22) PCT Filed: Feb. 17, 2006
(86) PCT No.: PCT/SE06/00219

§ 371 Date: Nov. 30, 2007

(30) Foreign Application Priority Data
Feb. 22, 2005 (SE) 0500410-6

(51) Int. Cl. A61B 5/00

(52) U.S. Cl. 600/424

(57) ABSTRACT

The present invention relates to an antenna system (1, 101, 201, 301, 401, 501) for tracking of a position of a target area (33, 133, 233, 333, 433, 533) within a body (5, 105, 205, 305, 405, 505), comprising a first antenna device (3, 103, 203, 303, 403, 503) and a second antenna device (9, 109, 209, 309, 409, 509), at least said second antenna device (9, 109, 209, 309, 409, 509) is to be located outside said body (5, 105, 205, 305, 405, 505), wherein an interface region (13, 113, 213, 313, 413, 513) between said body (5, 105, 205, 305, 405, 505) and at least said second antenna device (9, 109, 209, 309, 409, 509) at least partially consist of a dielectric medium having a relative permittivity closer to the permittivity of material within the body (5, 105, 205, 305, 405, 505) than the permittivity of air.

### TECHNICAL FIELD OF INVENTION

The present invention relates to an antenna system for tracking of a position of a target area within a living body, comprising a first antenna device to be located within said living body and a second antenna device to be located outside said living body.

### BACKGROUND OF THE INVENTION

Patients with e.g. cancer tumors are often treated with radiotherapy, usually delivered as a plurality of fractions over a period of time. The plurality of fractions that are used for the treatment serves to reduce the risk of side effects that may be caused by the treatment.

Due to organ and patient motions during the treatment, as well as between the treatments, resulting from respiration, blood flow, gastric motions, and other causes, it is often required to add a specific margin around the target area in which the radiotherapy energy should be projected, in order to keep a good treatment efficiency. This requirement may cause that the healthy organs around the target will be affected by high energy radiation, thus increasing the risk for side effects as a result.

In order to reduce the risk of side effects during radiotherapy treatment, different kind of target area positioning devices are often used. For instance, a target area of the tumor may be monitored during the treatment in order to enhance the positioning of the delivery of the radiotherapy.

WO02/100485 discloses a system and a method for locating and tracking the position of a tumor within a body by means of a beacon located inside the body of a patient. The described system and method determine the position of the beacon by measuring the distance to the beacon from several sensors by registering an absolute signal strength from the beacon.

### SUMMARY OF THE INVENTION

An object of the invention is to provide an alternative way of monitoring a target area within a living body.

The invention is, among other things, based on an insight that the monitoring of a target area within in a living body may be provided by establishing a communication channel between a first device located inside said living body and a second device located outside said living body for information exchange between said first device and said second device.

A first aspect of the invention relates to an antenna system for tracking of a position of a target area within a living body, comprising a first antenna device to be located within said living body and a second antenna device to be located outside said living body, wherein an interface region between said living body and said second antenna device at least partially consist of a dielectric medium having a relative permittivity closer to the permittivity of tissues within the living body than the permittivity of air.

A particular advantage of the antenna system according to the invention is that it enables for a proper establishment of a communication channel between said first antenna device and said second antenna device, which communication channel operate at a substantially low power level. Thereby, it is possible to use a signal, preferably a electromagnetic signal, with a measurable intensity at the same time as the transmission of said signal through the living body is substantially harmless. Accordingly, said tracking of a position of a target area may be achieved by arrangement of at least one communication channel between the first antenna device and the second antenna device.

An additional advantage of the antenna system according to the invention is that the reflection of the signal used for the communication between the first and second antenna device is substantially low as a result of a communication path between the first and second antenna device constituting a substantially equal permittivity. Accordingly, limited reflection of the signal used for the establishment of the communication channel results in a relatively low attenuation throughout the communication path.

It shall be noted that the first antenna device, to be located in a living body, as such do not interact with the living body. Hence, the antenna system may be applicable for other applications in which the first antenna device and the second antenna device is located in mediums with different permittivity. However, a preferred application is the monitoring of a target area within a living body.

A preferred field of application for the antenna system according to the invention is cancer treatment using radiotherapy, wherein a target area is monitored for the positioning of the radiation delivered from a radiotherapy arrangement. By the term “target area” is in this application meant a region of the living body that may be treated, measured or observed in any other way. Preferably, the antenna system according to the invention is integrated as a part of a treatment table of said radiotherapy treatment arrangement.

In one embodiment, said interface region has a relative permittivity substantially equal to the permittivity of a boundary surface of the living body. The term “boundary surface” is used to define the interface between a living body and the surrounding air. Accordingly, this may be the outer region of a living body, e.g. the skin of a human being, but may also be constituted by anything adjacent to the skin of a living body.

In an alternative embodiment, said interface region has a relative permittivity substantially equal to the permittivity of skin tissues of the living body, wherein the interface region preferably is placed adjacent to the skin of the living body at use. By using an interface region with a permittivity substantially equal to the boundary surface, i.e. an outer surface of the living body, the air gap between the second antenna device and the living body may be reduced. Accordingly, the reduced air gap between the boundary surface and the second antenna device reduce the amounts of reflections of the signal used for the establishment of the communication channel between the first antenna device and the second antenna device.

In one embodiment, at least a portion of said second antenna device constitutes at least a portion of said interface region. Suitably, the antenna device is provided with an end portion that is arranged to seat against the boundary surface of the living body. Preferably, the interface region is made of a relatively flexible material for adoption of the boundary surface shape when positioned close to the living body. Hence, the signal that is used for the establishment of said communication channel may be transmitted with low attenuation.

It is suitable to use an antenna device that enables for an operation with random polarized signals, which reduces polarization losses in communication systems operating in lossy mediums, such as the living body. A suitable antenna for random polarization is one with a circularly polarized radiation pattern. Preferably, the circular polarization is obtained by means of a helical antenna. Accordingly, said second antenna device advantageously comprises at least one helical antenna device.

According to one embodiment, the second antenna device at least partially consist of a dielectric medium having a relative permittivity substantially equal to the permittivity of said interface region. Suitably, the second antenna device is partially, or preferably fully, loaded with said dielectric medium. Filling the antenna with a dielectric medium provides for a boundary between the antenna and the interface region that reduce the amount of reflection of radiation used for the establishment of said communication channel between the first antenna device and the second antenna device.

In one embodiment said interface region comprises at least one interface device, such as a pillow device, wherein said interface device, in use, is placeable between said second antenna device and said living body. Hence, the interface device preferably has a relative permittivity closer to the permittivity of tissues within the living body than the permittivity of air. Alternatively, the interface device has a relative permittivity substantially equal to the permittivity of said boundary surface of the living body.

Further, the interface device is substantially conformable for adaptation of a shape essentially following a surface of said boundary surface of said living body. Accordingly, conditions with a close contact between said boundary surface and the interface device may be provided for a boundary that reduces the amount of reflections along the communication channel. Hence, a communication channel with a relatively low attenuation may be established between the first and second antenna device.

Advantageously, said interface device is adapted to be filled up with or drained from a medium for adaptation of its shape. The opportunity for variation of the shape of the interface device may be an advantage in medical applications where the patient with a substantially wide range of different body sizes may be treated. Preferably, the medium enclosed within the interface device having a relative permittivity closer to the permittivity of tissues within the living body than the permittivity of air.

In one embodiment the first antenna device is arranged as a medical implantable transmitter fixable relative to said target area within the living body. Advantageously, said first antenna device comprises a transmitter that is arranged to emit an electromagnetic signal adapted to preferably propagate with a frequency within the range of 5-1000 MHz. A phase difference of said electromagnetic signal is preferably detectable by the antenna system at a plurality of positions arranged outside the living body. Preferably said signal is detectable in at least three positions, preferably four, which positions are separated by a known distance. The detection of said phase difference may be used for determination of variations of a position of the first antenna device relative to the positions for detection outside the living body. Advantageously, each position for detection is arranged as a plurality of second antenna devices. In an alternative embodiment the position for detection may be provided as one second antenna device that are movable between the different positions.

The distance between the first antenna device and the second antenna device may be within an integer number n of wavelengths λ of the electromagnetic signal propagating in said living body, e.g. the distance may be between (n−1)*λ and n*λ. According to one embodiment the electromagnetic signal may propagate in said living body with a wavelength λ that is greater than the distance between the first and second antenna device. Further, said transmitter may be arranged to be energized by an external excitation source located outside the living body. It is suitably to provide the transmitter with a frequency converter, such as a mixer circuit. Said mixer circuit may receive and mix a first energizing signal with a first frequency and a second energizing signal with a second frequency for generation of said emitted electromagnetic signal. Said emitted electromagnetic signal from the transmitter preferably has a frequency that is substantially corresponding to the difference between said first and second frequencies.

According to at least one alternative embodiment, the transmitter of the first antenna device is arranged to be energized via a wire. In additionally at least one alternative embodiment, said first antenna device includes a source of energy for energizing of the transmitter.

Advantageously, said first antenna device and said second antenna device are arranged for both transmitting and receiving of an electromagnetic signal propagating through said living body.

In at least one embodiment, the first antenna device and the second antenna device, respectively, are arranged to transmit and/or receive an electromagnetic signal with a frequency between 5-1000 MHz, preferably 5-900 MHz, and especially 5-450 MHz. A particular advantage by using a frequency within the abovementioned range is that the emitted electromagnetic signal has a frequency which is able to propagate, e.g. through the tissues of a living body, with relatively low attenuation. Thereby, said electromagnetic signal, intended to preferably be detected and measured by the second antenna device, has a measurable signal intensity at the same time as a transmission through the living body which is substantially harmless.

According to at least one embodiment, the antenna system further comprises at least one dome arrangement, having a relative permittivity substantially equal to the permittivity of said interface region. The dome device is preferably shaped as a portion of a sphere.

Advantageously, the second antenna device having an aperture arranged to fit closely upon said dome device.

The second antenna device may be arranged movable over the spherical surface of the dome device in order to enable the second antenna device to be directed towards the first antenna device for establishment of the communication channel.

Advantageously, the antenna system is provided with a lens device for focusing of a radiation used for said communication channel. By use of such a lens device the phase and amplitude distribution can be controlled, which may be especially preferred in a low lobe antenna pattern. Preferably, said lens device is arranged within the second antenna device, and it shall be noted that the lens device may be located at any proper location within the second antenna device. Further, it shall be noted that the lens device may be provided as one single lens or a plurality of lenses. In at least one preferred embodiment, the lens device is provided as a single lens with a convex surface directed towards the boundary surface and a concave surface directed away from the boundary surface. The focal point established b said lens device may be relocated by partially moving or reshaping the lens device. In this way the thickness and/or the material of the lens may be varied, and hence the focal point will be relocated. By varying the material, the permittivity of the lens device may he changed, and thereby relocate of the focal point. In one embodiment the side of the lens directed to the boundary surface may be fixed and the side of the lens directed away from the boundary surface may be varied.

In accordance with one embodiment, said second antenna device is arranged as at least one ground plane helical antenna. Preferably, said helical antenna is arranged as a helicone antenna that is at least partially, or preferably fully, filled with a dielectric medium with substantially the same permittivity as said interface region.

In accordance with at least one embodiment, the second antenna device is arranged as an array comprising a plurality of antennas, such as dielectric loaded helical antennas. Advantageously, such an array of antennas works as a receiver for detecting and measuring a phase difference of an electromagnetic signal emitted from the first antenna device, in order to monitor a target area within a living body. Said array is preferably provided with at least three antenna devices, preferably four, separated by a known distance, wherein a three dimensional monitoring of said target area may be established. According to one embodiment, said array may be arranged as 4 antennas which together constitute a so called Y-shaped array. Accordingly, this Y-shaped array may comprise three antennas arranged as a circular pattern, spaced with an angle of 120° between each of the antennas, and a fourth antenna that may be arranged in the middle of said circular pattern. For instance such an Y-shaped arrangement of the antennas may be an advantage regarding signal-to-noise (S/N) ratio etc.

By measuring the phase difference of the electromagnetic signal in at least four positions, the position of the first antenna device may be determined without knowledge of the phase generated at the first antenna device, by way of comparing the signal received at said at least four positions.

By arranging the antenna devices of the array with a known distance, the measured phase difference of the electromagnetic signal may he compared for determination of the first antenna device position relatively to the array of antenna devices. The use of an array comprising at least four positions for detection of the signal makes it possible to measure tie relation between said array and the first antenna device in three dimensions, wherein the position of the first antenna device, relatively to the array, may be monitored in real time. The determination of the first antenna device position can be made by way of triangulation, neural networks etc.

Further, it shall be understood that the position of the first antenna device may be monitored in three dimensions by an array comprising at least three positions for detection in the case of a first antenna device energized via a wire, wherein the phase of the signal emitted from the first antenna device is known.

In a preferred application, the determined position of the target area is used for positioning of a beam of a radiotherapy treatment arrangement.

In general, the electrical behavior of materials when they are subjected to electro magnetic (MG) fields is characterized by their constitutive parameters ∈ (permittivity or capacitivity), μ (permeability or inductivity) σ (conductivity), which in general are functions of applied field strength, position within the medium, direction of the applied field and frequency of operation.

It is to be noted that in this application the term “dielectric medium” is used to define a medium that is a poor conductor of electricity, but an efficient supporter of electrostatic fields.

Further, in this application the terms “permittivity”, or “capacitivity”, of the medium is used to define a parameter that indicates the relative (compared to free-space) charge (energy) storage capabilities of a dielectric medium.

### BRIEF DESCRIPTION OF THE DRAWINGS

By way of examples, embodiments of the invention will now be described with reference to the accompanying drawings in which:

FIG. 1 shows a schematic side view of a radiation therapy treatment arrangement, comprising an antenna system for monitoring of a target area within a living body.

FIG. 2 shows a schematic perspective view, partially in cross section, of an embodiment of an antenna system according to the invention.

FIG. 3 shows a schematic perspective view, partially in cross section, of an alternative embodiment of the antenna system according to the invention.

FIG. 4 shows a schematic perspective view, partially in cross section of yet an alternative embodiment according to the invention.

FIG. 5 shows a schematic perspective view, partially in cross section of an embodiment of an antenna system having two external antenna devices according to the invention.

FIG. 6 shows a schematic perspective view, partially in cross section of an alternative embodiment of the antenna system in FIG. 5.

### DETAILED DESCRIPTION OF PREFERRED EMBODIMENTS

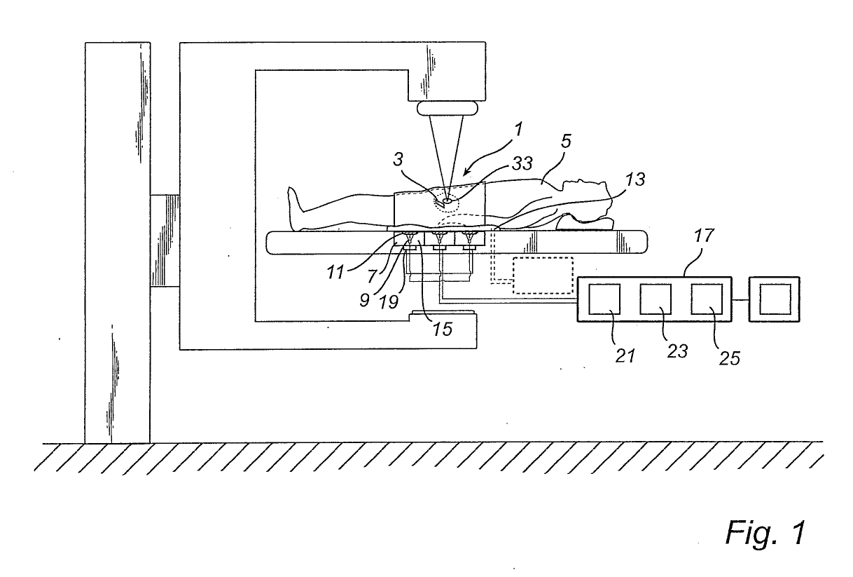

Embodiments of an antenna system 1 will now be described in more detail with reference to FIGS. 1-4.

According to FIG. 1, an antenna system 1 for communication with at least one medical implant 3 located within a living body 5 is shown. The antenna system 1 comprises an antenna array 7 with a plurality of positions for measurement, preferably provided as helical antennas 9, dome devices 11 and at least one interface device 13. The antenna array 7 is composed by a plurality of antenna scanning cells 15, wherein the antenna device 9 of each antenna scanning cell 15 may be moved over the surface of said dome device 11 and thereby changing the rotational arc for alignment of each antenna device 9 towards the medical implant 3.

Additionally, the antenna system 1 according to FIG. 1 comprises an antenna system control unit 17. Preferably, each antenna scanning cell 15 has a cell scanning control unit 19 that determine at which solid angle a beam of a RF (Radio Frequency) signal is immersed in the living body 5. The determination of the solid angle of the immersed beam enabling that the antenna position in relation to a system reference point may be determined.

Further each cell scanning control unit 19 is connected to said antenna system control unit 17 by means of a connection device. The antenna system control unit 17 comprises a scanning hardware 21, for controlling of the antenna devices 9 of the antenna scanning cells 15, and a RF unit 23, for transmitting and/or receiving said RF signals. The RF signals are generated, amplified or modulated in the RF unit 23 in order to control the shape, intensity and form of the beam of the RF signal.

As shown in FIG. 1, the antenna devices 9 of each antenna scanning cell 15 are connected to the RF unit by means of a connection device. The RF unit 23 comprises transmitters and receivers, typical of a super-hetrodyne type, wherein a transmitted or received signal becomes available for processing. For instance, a received signal for processing are sent to a central processing unit (CPU) 25, wherein said signal is processed, for instance by means of averaging and integration for improvement in the signal-to-noise (S/N) ratio. After processing, the signal may be used for creation of information of a scan view, a pictorial view or a three dimensional (3D) position of the source on a terminal not shown), or the like, used by an operator.

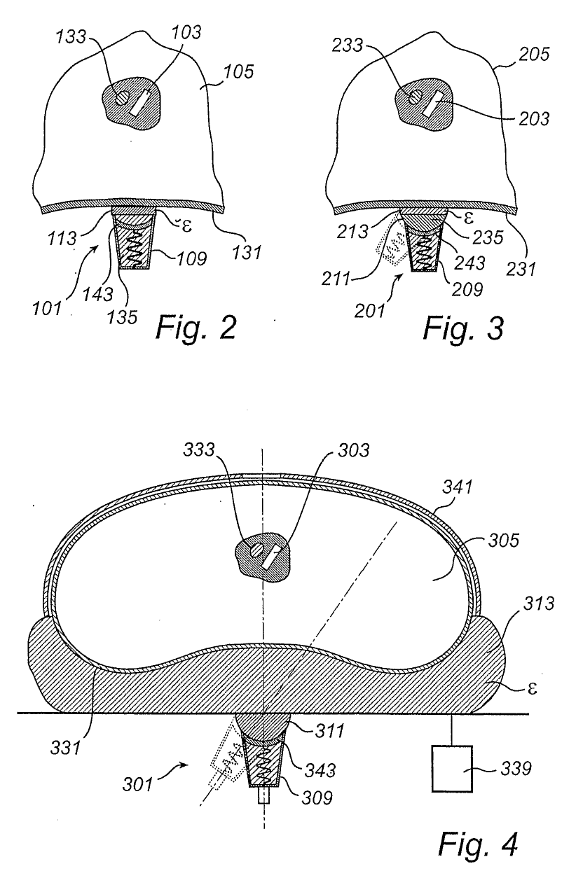

The antenna system 101 according to FIG. 2 comprises a helical antenna 109 to be arranged in close contact with a boundary surface 131 of a living body 105, such as the skin of a human body. In use, the helical antenna 109 is arranged at the outside of said living body 105. A medical implant 103 is provided in a predetermined area within the living body 105. The implant 103 is utilized for monitoring of a target area 133 within the living body 105, such as a cancer tumor etc. The monitoring of the target area 133 is established by monitoring of the implant 103 position, wherein the position of the implant 103 relatively to the target area 133 may be predetermined based on imaging data taken with an imaging system, such as a CT-scan (Computer Tomography-scan), MRI (Magnetic Resonance Imaging), Ultrasound, PET (Positron Emission Tomography) or the like.

The medical implant 103, which is fixable relative to a target area 133 within a living body 105, preferably comprise a transmitter arranged to emit an electromagnetic signal adapted to propagate with a frequency, wherein said frequency may be within the range of 5-1000 MHz so that a phase difference of said electromagnetic signal in at least three positions, preferably four, separated by a known distance is detectable by the second antenna device 109 for tracking variations of a position of the implant 103 relatively to said second antenna device 109. Preferably, said electromagnetic signal is adapted to propagate with a frequency within the range of 5-900 MHz, and particular within the range of 5-450 MHz. Preferably, the wavelength of the electromagnetic signal propagating in said living body 105 is greater than the distance between the implant 103 and the second antenna device 109.

A RF signal emitted from the implant 103 may be fed into the helical antenna 109 by means of a feeding device and travels through the helical antenna 109, wherein said RF signal may be radiated as a spherical beam around a beam axis.

The helical antenna 109 is at least partially, or preferably fully, filled with a dielectric medium 135 having a relative permittivity ∈ closer to the permittivity of tissues within the living body than the permittivity of air.

An end portion of the helical antenna 109, closest to the boundary surface 131 of the living body 105, constitutes an interface region 113 to be applied in close contact with said boundary surface 131. Said interface region 113 consist at least partially, or fully, of a dielectric medium 135 having a relative permittivity ∈ closer to the permittivity of tissues within the living body 105 than the permittivity of air. In this way a preferred communication channel may be established between said helical antenna 109 and said implant 103, wherein the RF signal may pass the boundary surface 131 of the living body 105 with a preferably low reflection, and thereby a relatively low attenuation.

The embodiment of the antenna system 201 according to FIG. 3 comprises a helical antenna 209 and an implant 203 to be arranged within a living body 205 in relation to a target area 233. Further, the embodiment shown in FIG. 3 comprises a dome device 211 to be arranged between said helical antenna 209 and a boundary surface 231 of the living body 205. The dome device 211 is preferably shaped as a portion of a sphere. Advantageously, the helical antenna 209 having an aperture arranged to fit closely upon said dome device 211, wherein the helical antenna 209 may be moved around over the surface of the dome device 211. The movement of said helical antenna 209 permits the RF beam of the antenna system 201 to be aimed towards the implant 203 by movement of said second antenna device 209 over an angle of scanning.

Accordingly, the helical antenna 209 becomes maneuverable in order to direct the beam in a range of directions, wherein the second antenna device 209 may be aimed towards the implant 203 within the living body 205.

The dome device 211 is partially, or preferably fully, filled with a dielectric medium 235 having a relative permittivity ∈ closer to the permittivity of tissues within the living body than the permittivity of air. An end portion of the dome device 211, closest to the boundary surface 231 of the living body 205, constitutes an interface region 213 to be applied in close contact with said boundary surface 231. Said interface region 213 consists at least partially, or fully, of a dielectric medium with a relative permittivity ∈ closer to the permittivity of the tissues within the body than the permittivity of air. The interface region 213 may be arranged as an integral portion of the dome device 211 or as a separate part arranged in close contact with the dome device 211.

According to one embodiment, the second antenna device 209 and the dome device 211 is filled at least partially, preferably fully, with a medium having the same permittivity ∈ as the permittivity ∈ of the interface region 213.

The embodiment of the antenna system 301 according to FIG. 4 comprises a helical antenna 309 and an implant 303 to be arranged within a living body 305. Further, the embodiment shown in FIG. 4 comprises a dome device 311 of a similar type as shown in FIG. 3, said dome device 311 is used for the aiming of the second antenna device 309 towards said implant 303.

Additionally, the embodiment of the antenna system 301 according to FIG. 4 comprises an interface region provided as an interface device 313 for achievement of a desired smooth interface against the boundary surface of the living body 305. The interface device 313 is substantially conformable for adoption of a shape which essentially may follow a surface of a boundary surface 331 of the living body 305. The interface device 313 is intended to be arranged between the dome device 313 and the boundary surface 331 of the living body 305 during use of the antenna system 301. According to an alternative embodiment (not shown), which not comprising a dome device 311, the interface device 313 may be provided in contact with the helical antenna 309 and said boundary surface 331.

Preferably, said interface device 313 is provided as a substantially soft pillow device that substantially matches the permittivity of the living body. Especially, said interface device 313 at least partially, or preferably fully, constitutes of a dielectric medium with a permittivity ∈ closer to the permittivity of tissues within the living body than the permittivity of air.

The softness or flexibility of the interface device 313 enables it to take the adaptable form of the boundary surface 331 and the adaptable form of the surface of the dome device 311, or helical antenna 309, closest to the boundary surface 331. Advantageously, the interface device 313 is filled with said dielectric medium having the permittivity ∈ closer to the permittivity of tissues within the living body 305 than the permittivity of air. Accordingly, the interface device 313 is filled with a medium with a relative permittivity substantially equal to the permittivity of the dome device 311, and/or second helical antenna device 309, wherein the interface device 313 matches the surrounding mediums for establishment of a communication channel with preferably low interface reflections of the radiation used for the communication channel between the helical antenna 309 and the implant 303.

The interface device 313 constitute a media matching interface region, preferably provided as a pillow device, which may be filled with a dielectric medium under pressure, preferably supplied together with said dielectric medium from a supplying device, such as a pump 339. Such a pillow device 313 is preferably filled up before establishing the communication channel between the helical antenna 309 and the medical implant 303 within the living body 305. The filling of the pillow device 313 may be continued until the pillow device 313 takes the shape of the boundary of the surrounding objects, such as the boundary surface 331 and the dome device 311. However, the softness of such a media matching interface region permits it to take the conformable form of the boundary surface 331 of the living body without additional efforts.

According to one embodiment, the interface device 313 is provided with free ends 341, such as a belt, which may be placed around the living body 305 and secured to each other. The attachment of the free ends 341 around the living body 305 may bring said body into a substantially fix position in relation to the interface device 313, and accordingly in relation to a treatment table on which the patient's body may be placed during treatment.

The at least partially dielectric filled helical antenna 109, 209, 309 shown in FIGS. 2-4 may comprise a lens 143, 243, 343 for enabling that a focal point of the antenna beam may be positioned at a point deep within the living body in order to reach the implant 103, 203, 303. The information required to determine whether the focal point has reached the implant 103, 203, 303 or not may be determined by measuring a signal emitted from said medical implant 103, 203, 303 by means of the helical antenna device 109, 209, 309. The measurement of said signal is executed during movement of the focal point in order to scan for a suitable communication between the first 103, 203, 303 and second antenna device 109, 209, 309. The position of the focal point is established when the communication between the medical implant 103, 203, 303 and the helical antenna 109, 209, 309 is within a proper rate, e.g. close to when a maximum signal strength is achieved For an array comprising a plurality of antenna devices 109, 209, 309 this procedure is repeated for each antenna device 109, 209, 309.

Preferably, the embodiments of the antenna system according to FIGS. 1-4 comprise a helical antenna device 109, 209, 309 that is at least partially, preferably fully, loaded with a dielectric medium. Advantageously, said helical antenna 109, 209, 309 is arranged as a ground plane helical antenna. Further, it is preferred to arrange said helical antenna 109, 209, 309 as a so called helical conical antenna, for better antenna system performance. Such a helical conical antenna 109, 209, 309 is also known as a helicone antenna.

An antenna system 1, 101, 201, 301 comprising a helicone antenna 9, 109, 209, 309 may be used in a multilateration position location system that utilize measurements from at least three, preferably four, or more antenna devices 9, 109, 209, 309 in order to determine the three dimensional (3D) position of a medical implantable transmitter 3, 103, 203, 303.

According to an alternative embodiment, a dynamic antenna system 1, 101, 201, 301 may be achieved by having one single antenna device 9, 109, 209, 309 that can be moved back and forth under the living body in order to form a specific arrangement, which functionality may be compared with the functionality of an antenna system comprising a plurality of fix antenna devices. Thus, one movable single antenna device of such a dynamic antenna system may undertake the function of a static antenna system 1, 101, 201, 301 with an array of antennas, comprising a plurality of fix antenna devices 9, 109, 209, 309, during a predetermined period of time. In the dynamic antenna system formation, the single antenna device 9, 109, 209, 309 preferably could be moved in three dimensions between positions for detection of the signal of the communication beam. Additionally, the use of one single dynamic antenna device is a good possibility since mutual coupling between antenna devices is eliminated, which in some specific applications may give a reduced accuracy of the measurements conducted by means of an antenna array 7. Further, it shall be noted that said dynamic antenna system 1, 101, 201, 301 may be provided as a plurality of movable antenna devices 9, 109, 209, 309.

However, it shall be noted that the antenna system formation preferably may be arranged movable regardless of the antenna system formation, i.e. dynamic or static antenna system formation. Accordingly, in treatment sessions, the antenna devices 9, 109, 209, 309 may be moved away under the patient after the implant positioning, in order to avoid that the antenna devices 9, 109, 209, 309 will be located in the path of an incoming radiation beam used in cancer tumor treatments.

According to an alternative embodiment, movement of the focal point is utilized for the determination of the position of the first antenna device 3, 103, 203, 303 when located within the human body 5, 105, 205, 305. The communication between the first 3, 103, 203, 303 and second antenna device 9, 109, 209, 309 may be scanned by moving the focal point until a considerable good communication is achieved. When said considerable good communication is achieved the position of the first antenna device 3, 103, 203, 303 in relation to the second antenna device 9, 109, 209, 309 may be determined based on information of the angle of the beam and the location of the focal point in relation to the second antenna device 9, 109, 209, 309. By repeating this procedure for a series of antenna devices 9, 109, 209, 309 located at different pre-determined locations, a three dimensional monitoring of the medical implant 3, 103, 203, 303 may be achieved. The information regarding the position of the medical implant 3, 103, 203, 303 is used for an alternative determination of the position of a target area 33, 133, 233, 333, such as a tumor, within the living body 5, 105, 205, 305, which target area 33, 133, 233, 333 for instance may be treated by means of a radio therapy arrangement.

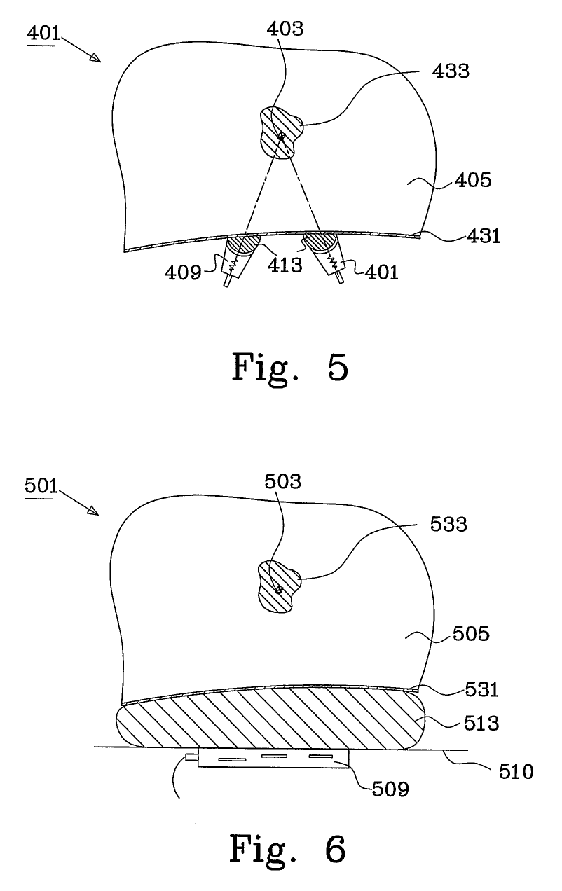

In addition to the embodiments described in connection with FIGS. 1-4, it is possible to implement the invention using two externally arranged antenna devices and a reflector, which is fixable relative the target area. FIGS. 5 and 6 will be used to describe two different embodiments to illustrate this.

The antenna system 401 according to FIG. 5 comprises a transmitting helical antenna 401 and a receiving helical antenna 409 provided with an interface region 413 to be arranged in close contact with a boundary surface 431 of a non-living body 405, such as a container filled with a dielectric material. In use, the helical antennas 401 and 409 are arranged at the outside of said container 405. An item 403 that will reflect electromagnetic signals, such as a piece of metal or radar reflector, is provided in a predetermined area within the container 405. The reflecting item 403 is utilized for monitoring of a target area 433 within the container 405. The monitoring of the target area 433 is established by monitoring of the position of the reflective item 403, wherein the position of the reflective item 403 relatively to the target area 433 may be predetermined based on imaging data taken with an imaging system as previously described in connection with FIG. 1. The transmitting 401 and receiving antenna device 409 may be of any type as have been described above, and the figure merely describe the principal function of the embodiment with two externally arranged antenna devices and a reflecting item arranged close to a target area.

The transmitting antenna 401 and the receiving antenna 409 are at least partially, or preferably fully, filled with a dielectric medium having a relative permittivity ∈ closer to the permittivity of the internal material inside the container than the permittivity of air.

FIG. 6 shows an embodiment of an alternative antenna system similar to the antenna system described in connection with FIG. 5. The antenna system 501 according to FIG. 6 comprises a combined transmitting and a receiving waveguide aperture antenna 509 to be arranged in close contact with an interface device 513 arranged between the antenna 509 and a boundary surface 531 of a living body 505, as illustrated in connection with FIG. 4. In use, the combined transmitting and receiving antenna 509 is for instance arranged in a treatment table 510. An item 503 that will reflect electromagnetic signals, such as a piece of metal or radar reflector, is provided in a predetermined area within the living body 505. The reflecting item 503 is utilized for monitoring of a target area 533 within the living body 505. The monitoring of the target area 533 is established by monitoring of the position of the reflective item 503, wherein the position of the reflective item 503 relatively to the target area 533 may be predetermined based on imaging data taken with an imaging system as previously described in connection with FIG. 1.

The combined transmitting and receiving antenna 509 is at least partially, or preferably fully, filled with a dielectric medium having a relative permittivity ∈ closer to the permittivity of tissues within the living body than the permittivity of air. Furthermore, the combined transmitting and receiving antenna 509 may be transmitting electromagnetic signals for a predetermined time period, and thereafter receiving the radar echo from the reflective item 503 within the living body.

Other types of antennas, such as a horn antenna, may be used instead of helicone antennas or waveguide aperture antennas as described in connection with FIGS. 1-6. The selected type of antenna is not essential for the invention as such, but the selected type of antennas is essential when creating the communication channel between the two antenna devices.

1. Antenna system for tracking of a position of an inserted object arranged relative to a target area within a body, said system is configured to detect the position of the inserted item and comprises a first antenna device and a second antenna device, at least said second antenna device is to be located outside said body, wherein an interface region between said body and at least said second antenna device at least partially consist of a dielectric medium having a relative permittivity closer to the permittivity of material within the body than the permittivity of air.

2. Antenna system according to claim 1, wherein said tracking of a position of a target area is achieved by arrangement of at least one communication channel between the first antenna device and the second antenna device.

3. Antenna system according to claim 1 wherein said interface region has a relative permittivity substantially equal to the permittivity of a boundary surface of the body.

4. Antenna system according to claim 1, wherein said interface region has a relative permittivity substantially equal to the permittivity of surface materials of the body.

5. Antenna system according to claim 1, wherein at least a portion of at least said second antenna device constitutes at least a portion of said interface region.

6. Antenna system according claim 1, wherein said at least second antenna device comprises at least one helical antenna device.

7. Antenna system according to claim 1, wherein at least said second antenna device at least partially consists of a dielectric medium having a relative permittivity substantially equal to the permittivity of said interface region.

8. Antenna system according to claim 1, wherein said interface region comprises at least one interface device, such as a pillow device, wherein said interface device, in use, is placeable between at least said second antenna device and said body.

9. Antenna system according to claim 8, wherein said interface device is substantially conformable for adoption of a shape essentially following a surface of said boundary surface of said body.

10. Antenna system according to claim 8, wherein said interface device is arranged to be filled up with or drained from a medium for adoption of its shape.

11. Antenna system according to claim 1, wherein said first antenna device is to be located within said body.

12. Antenna system according claim 1, wherein said inserted object is the first antenna device is arranged as an implantable transmitter fixable relative to said target area within the body.

13. Antenna system according to claim 1, wherein said first antenna device is to be located outside the body wherein an interface region between said body and said first antenna device at least partially consist of a dielectric medium having a relative permittivity closer to the permittivity of material within the body than the permittivity of air.

14. Antenna system according to claim 13, wherein said inserted object is a reflector arranged to be fixable relative to said target area within the body.

15. Antenna system according to claim 13, wherein said first antenna device is integrated into said second antenna device.

16. Antenna system according to claim 1, wherein said first antenna device and said second antenna device, respectively, are arranged to transmit and/or receive an electromagnetic signal with a frequency between 5-1000 MHz, preferably 5-900 MHz, and especially 5-450 MHz.

17. Antenna system according to claim 1, wherein the antenna system further comprises at least one dome device having a relative permittivity substantially equal to the permittivity of said interface region.

18. Antenna system according to claim 17, wherein said second antenna device has an aperture arranged to fit upon said dome device.

19. Antenna system according to claim 1, wherein the antenna system is provided with a lens device for focusing of a radiation beam established between the first antenna device and the second antenna device.

20. Antenna system according to claim 1, wherein said second antenna device is arranged as at least one ground plane helical antenna at least partially filled with a medium having substantially the same permittivity as the interface region.

21. Antenna system according to claim 1, wherein said second antenna device is arranged as at least one waveguide aperture antenna at least partially filled with a medium having substantially the same permittivity as the interface region.

22. Antenna system according to claim 1, wherein said body is a living body, having tissue within the living body and skin tissue at the surface.

23. Antenna system according to claim 1, wherein said body is a non-living body.
